(19) United States
(12) Patent Application Publication
(10) Pub. No.: US 2007/0049888 A1
(43) Pub. Date: Mar. 1, 2007

(54) Absorbent core comprising a multi-microlayer film

(76) Inventors: Dave Allen Soerens, Neenah, WI (US); Vasily A. Topolkaraev, Appleton, WI (US); Duane Girard Uitenbroek, Little Chute, WI (US); Patrick Martin Downey, Appleton, WI (US)

Correspondence Address: KIMBERLY-CLARK WORLDWIDE, INC., 401 NORTH LAKE STREET, NEENAH, WI, 54956, US

(21) Appl. No.: 11/217,587

(22) Filed: Aug. 31, 2005

(51) Int. Cl. A61F 13/15

(52) U.S. Cl. 604/372; 604/367

(57) ABSTRACT

An absorbent core comprises a multi-microlayer film having a plurality of coextruded microlayers that can perform the multiple functions of acquisition, retention and distribution of fluids. The multi-microlayer films may be formed in a coextrusion process. Each microlayer is manufactured from either a high-recovery polymer or a low-recovery polymer and may be in a substantially alternating configuration within the film. A disruptive force, such as a stretching force, can be applied to at least one portion of the multi-microlayer film to partially delaminate the low-recovery microlayer from the high-recovery microlayer to form corrugations in that portion. The multi-microlayer film can be apertured and additives may be included in either polymer layer.

### BACKGROUND

Disposable absorbent products currently find widespread use in many applications. For example, in the infant and child care areas, diapers and training pants have generally replaced reusable cloth absorbent articles. Other typical disposable absorbent products include, but are not limited to, feminine care products such as sanitary napkins or tampons, adult incontinence products, and health care products such as surgical drapes or wound dressings. A typical disposable absorbent product generally comprises a composite structure which can include a liquid-permeable topsheet, a fluid acquisition layer, a fluid distribution layer, a fluid retention layer, and a liquid-impermeable backsheet. These products often additionally include some type of fastening system for fitting the product onto the wearer.

In addition, there is an on-going desire to make absorbent articles more “underwear-like” in both the dry and wet condition. Absorbent articles, such as sanitary napkins for example, are intended to capture and hold fluid while maintaining a discreet feel. However, with an insult of a relatively large volume, an absorbent article can only maintain a discreet and underwear-like fit and feel if the fluid is spread over a large portion of the garment area. Extensive distribution of the fluid over the widest possible area allows the garment to retain the insult fluid with a minimum increase in garment thickness, thereby maintaining a more underwear-like fit in the wet condition. Therefore, there is a need for a thin absorbent core which can provide multiple functions, such as acquisition, retention and distribution of fluids to help maintain a thin garment having an underwear-like feel, even when wet, to make the user more comfortable and confident.

### SUMMARY

The present invention concerns an absorbent article, suitably a disposable absorbent article, such as a sanitary napkin. More particularly, the absorbent article comprises a topsheet, a backsheet and an absorbent core positioned between the topsheet and backsheet. Additionally, the absorbent core comprises a multi-microlayer film having a plurality of coextruded microlayers. Some of the microlayers comprise high-recovery polymers and others comprise low-recovery polymers, such that the layers substantially alternate within the multi-microlayer film. At least one section, or portion, of the multi-microlayer film can be disrupted by applying a disrupting force, for example a stretching force, such that the microlayers of a low-recovery polymer become partially delaminated from the microlayers of a high-recovery polymer in that portion and, upon removal of the disrupting force, can become corrugated within that portion. Accordingly, at least one portion of the multi-microlayer film should remain substantially non-disrupted such that the microlayers remain flat and substantially laminated to each other in that section. The multi-microlayer film may additionally be apertured to further improve absorbent properties.

Upon fluid insult of the absorbent core, fluid tends to enter the multi-microlayer film through the disrupted portion of the film via openings or apertures in the film. When the fluid comes into contact with the non-disrupted (e.g., unstretched) portion of the film, partial debonding of the microlayer laminate in that portion occurs in the direction of fluid flow (i.e., toward the distal ends of the film). This debonding action forms a capillary gradient within the non-disrupted portion of the film, which causes capillary pressures, which in turn create a suction of at least some of the fluid out of the disrupted portion and wicks the fluid through the non-disrupted portion to distribute the fluid into more remote locations of the absorbent article. The result is an absorbent core that can perform the multiple functions of at least acquisition, distribution and retention of fluids, which in turn results in an article that exhibits improved performance as well as greater comfort and confidence among the user.

Numerous other features and advantages of the present invention will appear from the following description. In the description, reference is made to exemplary embodiments of the invention. Such embodiments do not represent the full scope of the invention. Reference should, therefore, be made to the claims herein for interpreting the full scope of the invention.

### FIGURES

The foregoing and other features, aspects and advantages of the present invention will become better understood with regard to the following description, appended claims and accompanying drawings where:

FIG. 1 is a perspective view of one embodiment of an absorbent core that may be made in accordance with the present invention;

FIG. 2 is a top view of one embodiment of an absorbent core of the present invention demonstrating apertures in general;

FIG. 3 is a longitudinal cross-section view of one embodiment of an absorbent core of the present invention demonstrating particular types of apertures;

FIG. 4 is a plan view of a coextrusion system for making a microlayer polymer film in accordance with an embodiment of this invention;

FIG. 5 is a schematic diagram illustrating a multiplying die element and the multiplying process used in the coextrusion system illustrated in FIG. 4;

FIG. 6 is a representative, partially cut-away, top view of a body-facing side of an absorbent article which includes the absorbent core of the present invention as a component;

FIG. 7a is a top view of a Horizontal Wick Test Bed; and

FIG. 7b is a longitudinal side view of the Horizontal Wick Test Bed illustrated in FIG. 7a.

Repeated use of reference characters in the present specification and drawings is intended to represent the same or analogous features or elements of the present invention.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent article” generally refers to devices which can absorb and contain fluids. For example, personal care absorbent articles refer to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, and household/industrial absorbent articles.

The term “coform” is intended to describe a blend of meltblown fibers and cellulose fibers that is formed by air forming a meltblown polymer material while simultaneously blowing air-suspended cellulose fibers into the stream of meltblown fibers. The coform material may also include other materials, such as superabsorbent materials. The meltblown fibers containing wood fibers are collected on a forming surface, such as provided by a foraminous belt. The forming surface may include a gas-pervious material, such as spunbonded fabric material, that has been placed onto the forming surface.

The term “corrugations” includes, but is not limited to, corrugations, folds, pleats, and waves which occur in at least one of the microlayers of the present invention.

The term “disrupted portion” refers to a section of a multi-microlayer film that is less than the entire length of the film, and to which a disruptive force has been applied, for example a stretching force, to partially delaminate the polymer microlayers in that section, and which results in corrugations of at least one of the microlayers upon removal of the disruptive force.

The terms “elastic” and “elastomeric” are used interchangeably to refer to a material or composite that generally exhibits properties which approximate the properties of natural rubber. The elastomeric material is generally capable of being extended or otherwise deformed up to several hundred percent of elongation or more, and then recovering a significant portion of its shape, even to about its original length, after the extension or deforming force is removed.

The term “filled” refers to a polymer into which is incorporated particulate matter of specified size and composition, generally by melting the polymer and blending with the particulate matter in a suitable device.

The term “fluid impermeable,” when used to describe a layer or laminate, means that fluid such as water or bodily fluids will not pass substantially through the layer or laminate under ordinary use conditions in a direction generally perpendicular to the plane of the layer or laminate at the point of fluid contact.

The term “health/medical absorbent article” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The term “high-recovery polymer” refers to a polymer that, when in a solid state, exhibits elastic properties.

The term “household/industrial absorbent article” includes construction and packaging supplies, products for cleaning and disinfecting, wipes, covers, filters, towels, disposable cutting sheets, bath tissue, facial tissue, nonwoven roll goods, home-comfort products including pillows, pads, mats, cushions, masks and body care products such as products used to cleanse or treat the skin, laboratory coats, cover-alls, trash bags, stain removers, topical compositions, pet care absorbent liners, laundry soil/ink absorbers, detergent agglomerators, lipophilic fluid separators, and the like.

The term “insult target zone” refers to an area of an absorbent core where it is particularly desirable for the majority of a fluid insult, such as urine, menses, or bowel movement, to initially contact. In particular, for an absorbent core with one or more fluid insult points in use, the insult target zone refers to the area of the absorbent core extending a distance equal to 15% of the total length of the core from each insult point in both directions.

The term “low-recovery polymer” refers to a polymer, such as a thermoplastic melt-extrudable polymer, that exhibits stretchable and substantially non-elastic properties when in a solid state.

The term “materials” when used in the phrase “superabsorbent materials” refers generally to discrete units. The units can comprise particles, granules, fibers, flakes, agglomerates, rods, spheres, needles, particles coated with fibers or other additives, pulverized materials, powders, films, nanoparticles and the like, as well as combinations thereof. The materials can have any desired shape such as, for example, cubic, rod-like, polyhedral, spherical or semi-spherical, rounded or semi-rounded, angular, irregular, etc. Additionally, superabsorbent materials may be composed of more than one type of material.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

The term “melt-extrudable polymer” refers to a thermoplastic material having a melt flow rate (MFR) value of not less than about 0.2 grams/10 minutes, based on ASTM D1238.

The term “monolithic film” refers to a film that has multiple layers which adhere to one another and function as a single unit.

The term “multi-microlayer” means a film having a plurality of substantially alternating layers wherein, based upon the process by which the film is made, each microlayer becomes partially integrated, or adhered with, or laminated to the layers above and below the microlayer. This is in contrast to a “multi-layer” film wherein conventional co-extruded film-making equipment forms a film having only a few layers and wherein each layer is separate and distinct. Additionally, during formation of the films of the present invention, which includes applying a disruptive force to a portion of the multi-microlayer film, the microlayers will partially delaminate from one another, thereby permitting corrugations to be formed upon relaxation of the disruptive force or by activation of the film. However, partial integration or adherence of layers remains, unlike multi-layer films. The partial integration of the layers also increases the breathability, softness, tactility, and drapability of the film.

The term “non-disrupted portion” refers to a section of the multi-microlayer film of the present invention that is less than the entire length of the film, and to which a disruptive force, for example a stretching force, has not been applied such that the film in that section remains substantially laminated.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, airlaying processes, and bonded-carded-web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The term “partially delaminated” refers to a previously laminated composite wherein at least some, but not all, of the laminating bonds between the substrates have been broken.

The term “personal care absorbent article” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, pantiliners, panty shields, interlabials, tampons, and tampon applicators; adult-care products including wipes, pads such as breast pads, containers, incontinence products, and urinary shields; clothing components; bibs; athletic and recreation products; and the like.

The term “polymers” includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible configurational isomers of the material. These configurations include, but are not limited to isotactic, syndiotactic and atactic symmetries.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

The term “stretchable” refers to a material that is generally capable of being extended or otherwise plastically deformed, but which does not recover a significant portion of its shape after the extension or deforming force is removed.

The terms “superabsorbent” and “superabsorbent materials” refer to water-swellable, water-insoluble organic or inorganic materials capable of absorbing at least about 10 times their weight, or at least about 15 times their weight, or at least about 25 times their weight in an aqueous solution containing 0.9 weight percent sodium chloride. In contrast, “absorbent materials” are capable of absorbing at least 5 times their weight of an aqueous solution containing 0.9 weight percent sodium chloride.

These terms may be defined with additional language in the remaining portions of the specification.

### DETAILED DESCRIPTION

The present invention concerns an absorbent article, suitably a disposable absorbent article, such as a sanitary napkin. More particularly, the absorbent article comprises a topsheet, a backsheet and an absorbent core positioned between the topsheet and backsheet. Additionally, the absorbent core comprises a multi-microlayer film having a plurality of coextruded microlayers. Some of the microlayers comprise high-recovery polymers and others comprise low-recovery polymers, such that the layers substantially alternate within the multi-microlayer film. At least one section, or portion, of the multi-microlayer film can be disrupted by applying a disrupting force, for example a stretching force, such that the microlayers of a low-recovery polymer become partially delaminated from the microlayers of a high-recovery polymer in that portion and, upon removal of the disrupting force, can become corrugated within that portion. Accordingly, at least one portion of the multi-microlayer film should remain substantially non-disrupted such that the microlayers remain flat and substantially laminated to each other in that section. The multi-microlayer film may additionally be apertured to further improve absorbent properties.

Upon fluid insult of the absorbent core, fluid tends to enter the multi-microlayer film through the disrupted portion of the film via openings or apertures in the film. When the fluid comes into contact with the non-disrupted (e.g., unstretched) portion of the film, partial debonding of the microlayer laminate in that portion occurs in the direction of fluid flow (i.e., toward the distal ends of the film). This debonding action forms a capillary gradient within the non-disrupted portion of the film, which causes capillary pressures, which in turn create a suction of at least some of the fluid out of the disrupted portion and wicks the fluid through the non-disrupted portion to distribute the fluid into more remote locations of the absorbent article. The result is an absorbent core that can perform the multiple functions of at least acquisition, distribution and retention of fluids, which in turn results in an article that exhibits improved performance as well as greater comfort and confidence among the user.

The multi-microlayer films of the present invention provide a number of advantages as compared to conventional laminated films. For example, in addition to the improved fluid acquisition, retention, and distribution via capillary gradient in the structure, the films may also be softer, more drapable, and cushier.

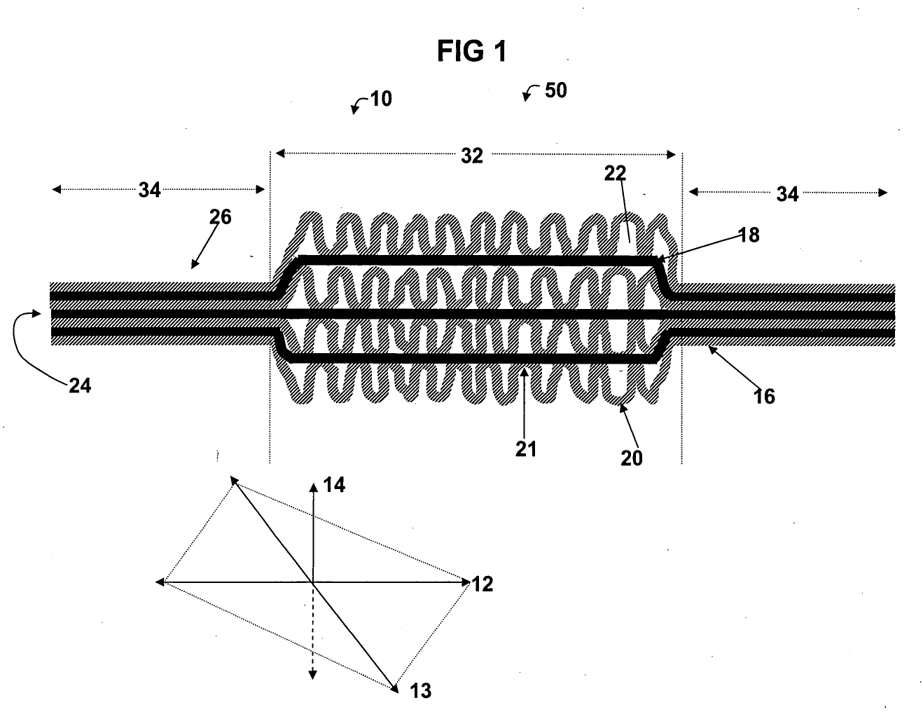

The present invention encompasses multi-microlayer films 10 that have sufficient elasticity, strength, breathability, and fluid handling properties for use in applications such as personal care absorbent articles, health/medical absorbent articles and household/industrial absorbent articles. FIG. 1 illustrates an absorbent core 50 of the present invention. More particularly, the absorbent core 50 comprising a multi-microlayer film 10 is illustrated in an opened and laid-flat state. The multi-microlayer film 10 defines a longitudinal direction 12 and a lateral direction 13 that is perpendicular to the longitudinal direction 12. The multi-microlayer film 10 also defines a z-direction 14 that is perpendicular to the plane formed by the longitudinal direction 12 and the lateral direction 13.

The multi-microlayer film 10 of the present invention comprises a plurality of coextruded microlayers 24 which form a laminate structure 26. The coextruded microlayers 24 include a plurality of high-recovery polymer microlayers 18 comprising an elastomeric, melt-extrudable polymer and a plurality of low-recovery polymer microlayers 16 comprising a stretchable melt-extrudable polymer capable of forming corrugations. The plurality of high-recovery polymer microlayers 18 and the plurality of low-recovery polymer microlayers 16 can be arranged in a series of parallel repeating laminate units. Each laminate unit comprises at least one of the low-recovery polymer microlayers 16 and at least one of the high-recovery polymer microlayers 18. Desirably, each laminate unit has one of the low-recovery polymer microlayers 16 laminated to one of the high-recovery polymer microlayers 18 so that the coextruded microlayers 24 substantially alternate. Then, after applying and releasing a disruptive force to a portion 32 of the multi-microlayer film 10, corrugations 20 can form in that portion 32 in the low-recovery polymer microlayers 16, the high-recovery polymer microlayers 18, or both. These corrugations 20 can produce channel spaces 22 between the layers 16,18 in the disrupted portion 32. These channel spaces 22 provide capillary voids for fluid absorption and wicking in the film 10. In addition, activation using heat or microwave energy can be used to help form or enhance the corrugations 20 in the partially delaminated portion 32 of the multi-microlayer film 10.

The microlayers 16,18 of the present invention form laminate films 10 with high integrity and strength because the layers 16,18 partially integrate into each other to form strong adhesion between the layers, which in turn reduces or eliminates complete delamination after coextrusion, in contrast to What would typically be observed from conventional co-extruded multi-layer films. Indeed, even after stretching a portion 32 of the multi-microlayer film 10, only partial delamination occurs, rather than complete delamination, which results in the formation of corrugations 20, as described above. Accordingly, the microlayers 16,18 of the present invention enable combinations of two or more layers of normally incompatible polymers into a monolithic film with strong coupling between the individual layers.

The high-recovery polymer microlayers 18 of the present invention are desirably composed of a thermoplastic, melt-extrudable, elastomeric polymer. The function of the elastomeric layers 18 is to provide confinement and a sufficient contraction force to the low-recovery polymer microlayers 16 after a disruptive force has been applied to a portion 32 of the multi-microlayer film 10 to a specified draw ratio and is subsequently released. As such, the multi-microlayer film 10 desirably has substantially alternating microlayers of the high-recovery polymer and the low-recovery polymer.

Suitable high-recovery polymers include polyurethane elastomer, copolyether ester, polyether block polyamide copolymer, ethylene vinyl acetate (EVA) elastomer, styrenic block copolymer, olefinic elastomer, as well as other elastomers known to those skilled in the polymer art. Particularly desirable elastomeric resins include polyester polyurethane and polyether polyurethane. In one example, the elastomeric polymer resin was ESTANE 58245 polyurethane, available from Noveon, Inc., a business having offices located in Cleveland, Ohio, U.S.A. Other suitable commercially available elastomeric resins include PN 3429-219 and PS 370-200 MORTHANE polyurethanes, available from Huntsman Polyurethanes, a business having offices located in Chicago, Ill., U.S.A. and PEARLTHANE polyurethane, available from Merquinsa, a business having offices located in Boxford, Mass., U.S.A.

Still other suitable elastomeric materials include a polyether block polyamide copolymer, such as those commercially available in various grades under the trade designation PEBAX, available from Atofina Chemicals, Inc., a business having offices located in Birdsboro, Pa., U.S.A. Another elastomeric material includes copolyether-ester, such as those available in various grades under the trade designation ARNITEL, available from DSM, a business having offices located in Heerlen, Netherlands. Additional elastomeric materials include copolyether-ester sold under the trade designation HYTREL, available from Invista, a business having offices located in Wichita, Kans. U.S.A.

Shape memory polymers activatable by heat, radio frequency waves, or microwaves can also be utilized in the elastomeric component 18 of the multi-microlayer film 10. When a film 10 having such components is stretched, the latent deformation is preserved in the shape-memory elastomer layers. Heat or microwaves may activate the deformation as well as elastomer-layer contraction. As a result, microlayer corrugation and a significant expansion in the z-direction (film thickness) may be achieved by using heat or microwave activation. An example of a shape memory elastomer includes PEBAX 2533 resin (available from Atofina Chemicals, Inc.). The amount of heat used for activation depends on the specific materials used, but will generally be in the range of about 60° C. to about 120° C.

The low-recovery polymer microlayers 16 of the present invention are desirably composed of a thermoplastic, melt-extrudable polymer. Suitable low-recovery polymers are stretchable (but not substantially elastic) when in a solid state to allow a stretching of the disrupted portion 32 of the multi-microlayer film 10 and thereby formation of corrugations 20 upon removal of the force. Stretching in a solid state means stretching at a temperature below the melting point of the polymer. Stretching the disrupted portion 32 of the film 10 changes the dimensions of that portion 32 (discussed above) and may create porosity, thereby increasing the water vapor transport rate of the film, as well as breathability. The ratio of true tensile fracture stress (i.e., tensile force at failure divided by the cross-sectional area of the failed specimen), and the stress at yielding, is useful to determine the stretchability of the polymer film. In one aspect, the ratio of true tensile stress to the stress at yielding can be from about 1 to about 150, such as about 5 to about 100, or about 10 to about 50.

In addition, the low-recovery polymers may be biodegradable or non-biodegradable polymers. Biologically degradable polymers are characterized as being degraded in the presence of naturally occurring microorganisms such that the films break down into smaller pieces, or lose strength significantly, such that if the polymer film is placed in a biologically-active environment, for example composting and sludge digestion, the film will break down over time.

Suitable biodegradable polymers useful in the present invention include, but are not limited to, biodegradable aliphatic polyesters, polymers and copolymers of polycaprolactone, polymers and copolymers of polylactic acid (PLA), polymers and copolymers of polybutylene succinate, poly(butylene succinate-adipate), other biodegradable melt-extrudable polymers and copolymers, and blends and mixtures thereof. For example, these biodegradable resins include aliphatic-aromatic co-polyesters, such as EASTARBIO (available from Eastman Chemical Company, a business having offices located in Kingsport, Tenn., U.S.A.); aliphatic-aromatic co-polyester, such as ECOFLEX (available from BASF Corporation, a business having offices located in Mount Olive, N.J., U.S.A.); thermoplastic polyesteramides (such as those available from Baer Corporation, a business having offices located in Pittsburgh, Pa., U.S.A.); and melt-stable, semi-crystalline polylactic acid (PLA) polymers including LACTY (available from Shimadzu Corporation, a business having offices located in Kyoto, Japan), LACEA (available from Mitsui Chemicals, a business having offices located in Tokyo, Japan), NATUREWORKS (available from Cargil Dow LLC, a business having offices located in Minnetonka, Minn., U.S.A.), and L5000 and L9000 (available from Biomer, a business having offices located in Krailling, Germany), and those having a weight average molecular weight from about 50,000 g/mol to about 200,000 g/mol such as those disclosed in U.S. Pat. No. 5,773,562 to Gruber et al. entitled “Melt-Stable Semi-Crystalline Lactide Polymer Film and Process for Manufacture Thereof” which is incorporated herein by reference in a manner that is consistent herewith. Still other biodegradable resins include polyhydroxyalkanoates of varying composition and structure, and copolymers, mixtures and blends of the foregoing polymers.

Among biodegradable polymers, the polymers and copolymers of PLA are desired for this invention because of their stiffness, plasticization potential, ability to accept fillers, good processability, wettability, biodegradation, and other beneficial characteristics. Stretchability of PLA polymers and copolymers may be significantly improved when PLA is stretched above its glass transition temperature.

Non-biodegradable, melt-extrudable and stretchable low-recovery polymer microlayers may be formed from polyolefins, polyesters, and polyethers, as well as their copolymers and blends or mixtures thereof. Other examples include, but are not limited to, polypropylene (“PP”) and its copolymers, polyethylene and its copolymers, and blends or mixtures thereof. In other aspects, the polymer can be H 702-35NA polypropylene resin, available from Dow Chemical Company, a business having offices located in Midland, Mich., U.S.A. In one example, the low-recovery polymer was 3155 polyproylene resin (available from ExxonMobil Chemical Company, a business having offices located in Houston, Tex., U.S.A.) filled with molecular sieves and calcium carbonate. Non-biodegradable polymers may also include plasticizers, surfactants and other additives which may modify and improve the performance and processability of resulting films. Polypropylene is particularly useful in the present invention as a non-biodegradable polymer due to its stiffness, good processibility, ability to accept fillers and stretchability.

As alluded to above, the microlayers of low-recovery polymer 16 may also include processing additives and solid-state performance modifiers blended with the polymer (i.e., filled) in amounts from about 0.05 to 30 parts of additive to 100 parts of polymer resin. Suitable additives include a wide variety of materials such as surfactants, mineral acids, gelatin, adipic acid, metal halides, metal salts, polymeric acids, benzoic acid derivatives, glycol derivatives, phosphoric acid derivatives and sorbitan derivatives. Examples of still other suitable additives include, but are not limited to, polyethylene oxide, and polyethylene glycol. Antioxidants and ultraviolet stabilizers may also be added to improve oxidative stability and stability to UV light. The various additives can: have a plasticizing affect, improve melt flow characteristics, improve strength and toughness, improve modulus, modify crystalline structure, and control release properties.

The multi-microlayer film 10 may also include a third type of layer (not shown), such as a tie or transition layer, between the microlayers 16,18. This third layer can be useful for modifying or enhancing properties of the disrupted portion 32 of the multi-microlayer film 10 such as softness, opacity, fluid intake, fluid uptake and retention. The tie layer may also help control the debonding/delaminating between the low-recovery polymer microlayers 16 and the high-recovery polymer microlayers 18 in the disrupted portion 32, thus controlling the frequency and amplitude of the corrugations 20. The third component layer can also modify the adhesion between the high-recovery microlayers 18 and the low-recovery microlayers 16; provide response to fluids and other environments; prevent migration of plasticizers or other additives from one component layer 16,18 to another; and otherwise beneficially modify properties of the film 10.

The third microlayer can comprise a melt-processible polymer including: thermoplastic, water-soluble, water-swellable polymers such as superabsorbent materials; thermoplastic, melt-extrudable polyethylene oxide resins; or melt-extrudable polyvinyl alcohol resins. Other suitable polymers for the optional third component depend on the particular polymers used for the microlayers 16,18, but generally include filled polyolefins, thermoplastic polyesters, polyalkane-poly(ethylene oxide) block copolymers, filled polyester resins, polyethers, block copolymers of polyesters and polyethers, polyolefin block copolymers, ethylene vinyl acetate copolymers, and blends and mixtures thereof. The third microlayer can also include filler particles and/or other additives, such as surfactants, blowing agents, and the like.

As referenced above, a portion of the absorbent core 50 of the present invention can comprise corrugations. Corrugations 20 can be formed by applying a disruptive force to a section 32 of the multi-microlayer film 10, such as by applying a stretching force. When the disruptive force is subsequently released, the microlayers 16 become partially debonded from the high-recovery polymer microlayers 18. The retraction forces of the high-recovery polymer microlayers 18 create a “buckling” effect since at least some bonding 21 between the microlayers 16,18 remains in tact, but the low-recovery microlayers 16 are now greater in length than the high-recovery polymer microlayers 18 when in a retracted state. This “buckling” phenomenon results in the formation of corrugations 20 in one or both of the layers 16,18 and is a fundamental mechanism for the formation of the film 10 with corrugated microlayers. While corrugations 20 in the low-recovery microlayers 16 will tend to be more pronounced, the high-recovery polymer microlayers 18 may also form corrugations (not shown), but generally the channels formed will be in a direction substantially perpendicular to the channels 22 formed in the low-recovery microlayers 16. For example, if the stretching force is in the machine direction 12, the channels 22 will tend to be in the cross-machine direction 13. In addition, the channel spaces 22 in the high-recovery polymer microlayers 18 tend to be parallel to the stretching force. Accordingly, if channels 22 are formed in the cross-machine direction 13 of the low-recovery polymer microlayers 16, then channels may also be formed in the machine direction 12 of the high-recovery polymer microlayers 18. Consequently, drawability and sufficient stiffness to allow for buckling and the formation of corrugations 20 is a desired characteristic when selecting the microlayer polymer components.

The disrupted portion 32 of the multi-microlayer film 10 of the present invention may be pre-treated to prepare the portion for application of a disruptive force. The pre-treatment may be accomplished by annealing that portion 32 of the film at elevated temperatures, by spraying the portion 32 with a surface-active fluid (such as a liquid or vapor from the surface-active material employed to surface-modify the filler material or modify the components of the film), or by modifying the physical state of the portion 32 with ultraviolet radiation treatment, ultrasonic treatment, e-beam treatment, or high-energy radiation treatment. Pre-treatment may also include aperturing of the film, generation of z-directional or angular channels of varying size and shapes, which penetrate through the film thickness. In addition, the pre-treatment of the disrupted portion 32 of the multi-microlayer film 10 may incorporate a selected combination of two or more of the stretching techniques. Stretching techniques are known in the art. One such suitable stretching technique is disclosed in U.S. Pat. No. 5,800,758, which is incorporated herein by reference in a manner that is consistent herewith.

The parameters during application of the disruptive force, such as a stretching force, include stretching draw ratio, stretching strain rate, and stretching temperature. During stretching, a desired portion of the multi-microlayer film 10 may optionally be heated to provide a desired effectiveness of the stretching. In some aspects, the temperatures utilized during stretching may be in the range of from about 15° C. to about 100° C., such as about 25° C. to about 85° C. In one example, the temperature utilized during stretching was about 25° C.

In some aspects of the invention, the draw or stretching system may be constructed and arranged to generate a draw ratio which is not less than about 2 in the machine and/or cross-machine directions. The draw ratio is the ratio determined by dividing the final stretched length of the disrupted portion of the multi-microlayer film 10 by the original unstretched length of that same portion along the direction of stretching. In some aspects, the draw ratio in the machine direction (MD) can be not less than about 2, such as not less than about 2.5, or not less than about 3.0. In other aspects, the stretching draw ratio in the MD can be not more than about 16, such as not more than about 7.

Alternatively, it may be desirable to utilize stretching in the cross-machine direction (CD). In some aspects, the stretching draw ratio in the CD can be not less than about 2, such as not less than about 2.5, or not less than about 3.0. In other aspects, the stretching draw ratio in the CD can be not more than about 16, such as not more than about 7, or not more than about 5.

It may also be desirable to perform stretching in more than one direction, such with biaxial stretching. Biaxially stretching may be accomplished simultaneously or sequentially. In the case of sequential biaxial stretching, the initial stretching may be performed in either the MD or the CD.

In general, the film 10 of the present invention may have extensibility in the machine direction 12, in the cross-machine direction 13 or in both directions. In some aspects, the film 10 may have an extensibility of up to 100% elongation, such as about 50% elongation, and may retract to about its original length after the removal of a disruptive force.

As referenced above, the film 10 of the present invention may be made by using two materials having sufficiently different recovery percentages such that after stretching, the microlayers will form corrugations after release of the stretching force. As used herein, “recovery percentage” is defined as the percentage by which the film returns to its original shape. Accordingly, a film having a recovery percentage of 100 will return to its original shape and a film having a recovery percentage of 0 will not recover any of its pre-stretched shape.

The microlayers of a high-recovery polymer 18 can have a recovery percentage of about 30% to about 100%. The microlayers of a low-recovery polymer 16 can have a recovery percentage of from about 0% to about 50%. It is understood that the recovery percentage of the high-recovery polymer microlayers 18 should be higher than the recovery percentage of the low-recovery polymer microlayers 16 such that after stretching, partial delamination and recovery, the low-recovery polymer microlayers 16 will recover less of their original shape than the high-recovery polymer microlayers 18, and that the excess length of material will tend to form corrugations 20 in at least the low-recovery polymer microlayers 16. As the difference in the recovery percentages increases, more channel spaces 22 will be formed in the disrupted portion of the multi-microlayer film, and as the difference in the recovery percentages decreases, less channel spaces 22 will be formed in the disrupted portion.

The film 10 of the present invention can also have an increased void volume and significantly reduced bulk density in the disrupted portion 32, which results from the microlayer corrugations 20, which helps create void spaces between the corrugated microlayers. In one aspect, the disrupted portion 32 can have an increase in void volume of from about 500% to about 5000%, such as about 1000% to about 3000%, relative to the non-disrupted portion 34. In addition, the bulk density of the disrupted portion may be less than about 0.5 grams per cubic centimeter, such as less than about 0.1 grams per cubic centimeter, or less than about 0.05 grams per cubic centimeter, or less than about 0.03 grams per cubic centimeter.

The corrugations 20 in the disrupted portion 32 of the film 10 will generally be created by stretching and recovery of the microlayers 16,18. However, the corrugations may also be formed by activating the stretched multi-microlayer film. Activation may be done using an activating force, such as heat, electromagnetic radiation or humidity, depending on the materials used and the desired degree of corrugations in the post-processed film.

During the formation of corrugations 20, the disrupted portion 32 changes dimensions in the machine direction 12, the cross-machine direction 13 and in z-direction 14 (i.e., thickness direction). Typically, that portion 32 of the multi-microlayer film 10, after release of the disrupting force, tends to shrink in the cross-machine direction 13, expand slightly to moderately in the machine direction 12 and expand significantly in the z-direction 14, providing enhanced three-dimensional profiles and structures.

The thickness of the extruded microlayers can also have an impact on corrugation formation. In some aspects, each microlayer 16,18 can have a thickness from about 0.1 micron to about 150 microns, such as about 0.5 microns to about 100 microns. In other aspects, each microlayer 16,18 has a thickness of less than about 100 microns, such as less than about 50 microns. In still other aspects, each microlayer has a thickness of at least about 0.5 microns, such as at least about 1 micron.

The amplitude of the corrugations 20 in the disrupted portion 32 can range from about 5 microns to about 5 millimeters, such as about 10 microns to about 500 hundred microns. The width of the channel spaces 22 formed by the corrugations 20 may range from about 1 micron to about 10 millimeters, such as about 5 microns to about 5 millimeters, or about 10 microns to about 500 microns, or about 20 microns to about 250 microns. When the disrupted portion of the multi-microlayer film is insulted with a fluid, the tubular channel spaces 22 provide capillary voids for fluid absorption and wicking. The frequency of the corrugations 20 may be in the range of about 1 to about 10,000 per 10 millimeter of the film length, such as about 2 to about 2000 per 10 millimeter of the film length, or about 20 to about 1000 per 10 millimeter of the film 10 length. The amplitude and frequency of the corrugations 20 may be affected by one or more of the following factors: stiffness of the corrugated microlayers, thickness of the multi-microlayer film, thickness of the individual microlayers, the contraction force of the expanded elastomer layers, the number of microlayers forming the laminate, and/or the bonding and interaction between the layers. For example, the frequency may be increased and the amplitude of the corrugations may be reduced by increasing the number of microlayers or by reducing the multi-microlayer film thickness. In some aspects, multi-microlayer films 10 having a smaller thickness and a larger number of layers can provide smaller channel width. In other aspects, multi-microlayer films having a larger thickness and a smaller number of layers may provide larger channels and an increased amplitude of the microlayer corrugations.

As referenced above, disruptive forces, such as stretching operations, can be preformed in a uniaxial direction or biaxial direction. In addition, stretching operations may provide a microporous microlayer film with a distinctive porous microlayered morphology which may enhance water vapor transport through the film and may improve water access, enhance degradability of the film, and enhance elastomeric properties of the film. In some aspects, the film can be stretched from about 100 to about 1500 percent of its original length, such as about 100 to about 500 percent of its original length.

The corrugated-microlayers in the disrupted portion 32 of the multi-microlayer films 10 can be wettable and can convey, by capillary action, fluids through the film 10 in the machine direction 12, in the cross-machine direction 13, or in both directions.

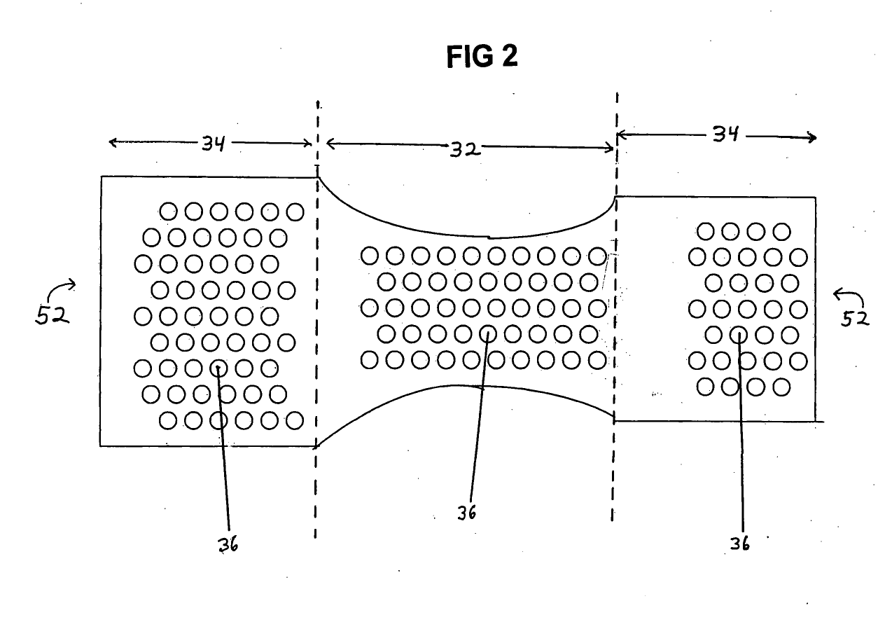

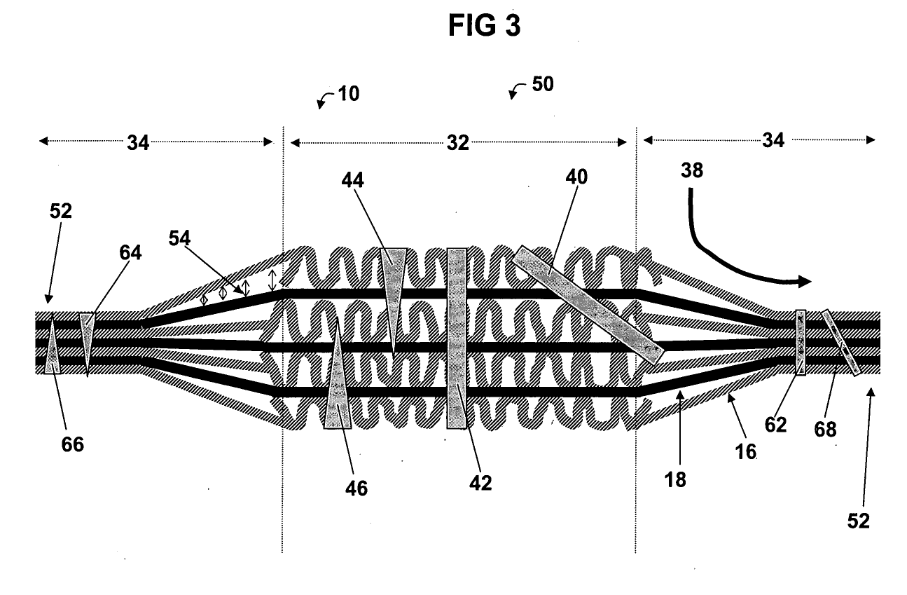

The disrupted portion 32 of the multi-microlayer film 10 is desirably located in an insult target zone of the absorbent article 50. The disrupted portion 32 can be apertured before or after application of the disruptive force. Referring to FIG. 2 and FIG. 3, the apertures 36 can provide one-way or two-way z-directional channels 38 or angular channels 40 for fluid access into the disrupted portion 32 to enhance fluid acquisition, adsorption, absorption and distribution functions, as well as fluid access out of the disrupted portion 32. Z-directional channels 42,44,46 will tend to direct fluids into or through the disrupted portion 32, while angular channels 40 will tend to direct fluids to specific areas, such as to the non-disrupted portion(s) 34 of the multi-microlayer film 10. Apertures 36 may also improve the vapor transport rate out of the disrupted portion 32. The apertures 36 may additionally facilitate or modify corrugation of the layers by creating strain-intensive sites around perforated channels. The aperture channels can penetrate through the entire thickness of the disrupted portion 32 to form complete apertures 42, or may only partially perforate the film to a specified depth to form fluid entrance channels 44 and fluid exit channels 46.

It has been discovered that the non-disrupted portion 34 of the multi-microlayer film 10 also plays an essential role in creating an improved absorbent core 50. As referenced above, upon fluid insult of the absorbent core, the majority of fluid tends to enter the disrupted portion 32 of the multi-microlayer film 10. However, the fluid can then be redirected by capillary action, through the channel spaces 22 and/or by the aperture channels 40,42,44,46 to the non-disrupted portion 34 of the multi-microlayer film 10. As the fluid comes into contact with the higher density non-disrupted portion 34, the microlayers 16,18 begins to debond in the direction of fluid flow 38 (i.e., towards the distal ends 52 of the multi-microlayer film 10). This action generates a capillary gradient 54 with very small capillaries at the fluid front providing suction from the larger capillaries formed by the debonding action of the microlayers 16,18. The result is that the fluid is drawn out of the disrupted portion 32 and is then distributed to more remote locations in the absorbent article, away from the insult target zone. This in turn prolongs the thinness and superior tactile feel of the absorbent article, while providing the user with greater comfort and confidence. The debonding of the microlayers 16,18 can be further enhanced through the use of fillers (such as, for example, molecular sieves and calcium carbonate) and/or surfactants within at least one of the microlayers, suitably the low-recovery polymer microlayer 18.

It may be desirable to aperture the non-disrupted portion 34 of the multi-microlayer film 10. One purpose of such apertures 62,64,66,68 is to provide an exit passage for the fluids as they wick through the non-disrupted portion 34. Additionally, they can direct the fluids to a specific location and into a different layer, if desired, such as an additional retention layer (not shown).

Accordingly, the multi-microlayer film 10 of the present invention may be apertured in the disrupted portion 32, the non-disrupted portion 34, or both. Aperturing the multi-microlayer film 10 may be accomplished by using numerous methods known in the art. For example, one such method includes punching holes using pins of varying diameter, density and configuration, which may optionally be arranged into a pattern desired for a specific application of the film. The pins utilized to punch holes and aperture the film may also be optionally heated. Other methods known in the art include, but are not limited to, high speed and intensity water jets, high intensity laser beams and vacuum aperture techniques. In some aspects, the microlayers 16,18 can have at least 2 apertures per square centimeter (cm²), such as at least 5 apertures per cm².

As referenced above, the polymers should be melt-extrudable so that the microlayers 16,18 can be coextruded to form the multi-microlayer film 10. In addition, the microlayers 16,18 can be permeable to fluid or water vapor when in the form of a film. Furthermore, at least one of the microlayer polymers is suitably selected such that the polymer is capable of being formed into a corrugated layer. By “capable of being formed into a corrugated layer” it is meant that after the non-elastomeric polymer and the elastomeric polymer are coextruded, stretching of the coextruded film will cause the non-elastomeric polymer to partially debond from the elastomeric layer such that when the stretching force is removed, the elastomeric polymer layer 18 recovers more of its original shape than the stretchable polymer layer 16, thereby causing corrugations to form 20. Since the polymers of the present invention are melt-extrudable, they have a melt flow rate (MFR) value. In some aspects, the melt-extrudable polymers have an MFR value of about 0.2 grams/10 minutes to about 100 grams/10 minutes, such as about 0.5 grams/10 minutes to about 50 grams/10 minutes, or about 5 grams/10 minutes to about 50 grams/10 minutes to provide desired levels of processibility. Additionally, the polymers may be wettable, which can increase the wicking properties of the film 10.

The number of microlayers in the multi-microlayer film 10 of the present invention can vary broadly from about 4 to about 100 in number, such as about 16 to about 60 in number, or about 17 to about 33 in number. However, based upon the thickness of each microlayer, the number of microlayers 16,18 in the film 10 is determined by the desired overall film thickness in both the disrupted portion 32 and the non-disrupted portion 34. In some aspects, the multi-microlayer film 10 can have a thickness in the disrupted portion 34 as measured under a 0.05 psi load of from about 0.5 mm to about 300 mm, such as about 2 mm to about 150 mm, or about 5 mm to about 75 mm. In one example, the thickness of the disrupted portion as measured under a 0.05 psi load was about 5 mm. In another example, the thickness of the disrupted portion as measured under a 0.05 psi load was about 6 mm. In other aspects, the non-disrupted portion 34 of the multi-microlayer film 10 can have a thickness as measured under a 0.05 psi load of from about 0.1 mm to about 25 mm, such as about 0.25 mm to about 13 mm, or about 0.5 mm to about 5 mm. In one example, the thickness of the non-disrupted portion as measured under a 0.05 psi load was about 0.12 mm. In another example, the thickness of the non-disrupted portion as measured under a 0.05 psi load was about 0.25 mm.

The relative thickness of the microlayers 16,18 of the multi-layer film 10 can be controlled by varying the feed ratio of the polymers into the extruders, thus controlling the constituent volume fraction. The multi-microlayer films suitably include between about 20% to about 90% by weight of high-recovery polymer resin and from about 80% to about 10% by weight of low-recovery polymer, such as between about 30% to about 70% by weight of high-recovery polymer resin and from about 70% to about 30% by weight of low-recovery polymer, or between about 50% to about 70% by weight of high-recovery polymer resin and from about 50% to about 30% by weight of low-recovery polymer. In one particular example, the multi-microlayer film comprised about 70% by weight high-recovery polymer resin and about 30% by weight low-recovery polymer.

The disrupted portion 32 of the film 10 can have a dramatically reduced tensile and shear modulus as compared to the non-disrupted portion 34. For example, in some aspects, the tensile modulus in the machine direction of the non-disrupted portion 34 can range from about 50 megapascals (MPa) to about 800 MPa, such as about 100 MPa to about 500 MPa. In comparision, the tensile modulus in the machine direction of the disrupted portion can range from about 0.2 MPa to about 50 MPa, such as about 0.3 MPa to about 20 MPa or about 0.5 MPa to about 5 MPa.

The polymers of the present invention may also include additives. Such additives include, but are not limited to, fillers, plasticizers, surfactants, solid-state modifiers, processing aids, and other additives to improve melt processability, reduce viscosity, enhance drawability/stretchability, improve surface properties and improve thermal stability. Surfactants and additives may additionally improve wettability and interactions with fluids. Furthermore, grafting, copolymerization, and modification of end groups may be used to modify the surface properties of the low-recovery polymer microlayers and their interaction with elastomeric layers.

Both the high-recovery microlayers 18 and the low-recovery microlayers 16 may include a supplemental material such as a filler material, a surfactant, or other surface active material. In some aspects, the filler material may be a particulate filler material to enhance water vapor permeability, thereby further increasing breathability. Particulate filler material creates discontinuity in the microlayers which provides pathways for water vapor to move through the film. Particulate filler material may also enhance the ability of the microlayer film to absorb or immobilize fluid, enhance biodegradation of the film, provide porosity-initiating debonding sites to enhance the formation of pores when a disruptive force is applied, improve processability of the microlayer film and reduce production costs.

Suitable filler materials may be organic or inorganic, and are desirably in a form of individual, discreet particles. Suitable inorganic filler materials include molecular sieves, zeolites, metal oxides, metal hydroxides, metal carbonates, metal sulfates, various clays, silica, alumina, powdered metals, glass microspheres, or vugular void-containing particles. Additional inorganic filler materials include calcium carbonate, barium sulfate, sodium carbonate, magnesium carbonate, magnesium sulfate, barium carbonate, kaolin, carbon, calcium oxide, magnesium oxide, aluminum hydroxide, and titanium dioxide. Still other inorganic fillers may include those with particles having higher aspect ratios such as talc, mica and wollastonite. In one example, molecular sieves and calcium carbonate were used as fillers for the low-recovery polymer.

Suitable organic filler materials include, for example, latex particles, particles of thermoplastic elastomers, pulp powders, wood powders, thermoplastic starch, cellulose derivatives such as carboxymethyl cellulose or hydroxypropyl cellulose, chitin, chitosan powder, powders of highly crystalline high melting polymers, beads of highly crosslinked polymers, organosilicone powders, and powders or particles of super absorbent polymers, such as polyacrylic acid and the like, as well as combinations and derivatives thereof.

In general, the filler materials may improve toughness, softness, opacity, vapor transport rate (breathability), biodegradability, fluid immobilization and absorption, skin wellness, and other beneficial attributes of the microlayer film. Particulate filler material is suitably present in the microlayer film in an amount from about 0.5 to about 70% by weight of the film. In some aspects, the average particle size of the filler material does not exceed about 200 microns. In other aspects, the average particle size of the filler does not exceed about 50 microns. In still other aspects, the average particle size of the filler does not exceed about 5 microns. In yet other aspects, the average particle size of the filler does not exceed about 1 micron.

Suitable commercially available filler materials include the following: SUPERMITE, an ultrafine ground calcium carbonate (available from Imerys, a business having offices located in Atlanta, Ga., U.S.A.) which has a top cut particle size of about 8 microns and a mean particle size of about 1 micron and may be coated with a surfactant, such as DOW CORNING 193 surfactant (available from Dow Corning, a business having offices located in Midland, Mich., U.S.A.) before mixing with the polymer; SUPERCOAT, a coated ultrafine ground calcium carbonate (available from Imerys) which also has a top cut particle size of about 8 microns and a mean particle size of about 1 micron; OMYACARB UF, high purity, ultrafine, wet ground calcium carbonate (available from OMYA, Inc., a business having offices located in Proctor, Vt., U.S.A.) which has a top cut particle size of about 4 microns and an average particle size of about 0.7 microns, and may also be coated with a surfactant such as DOW CORNING 193 surfactant before mixing with the polymer; and OMYACARB UFT calcium carbonate, an ultrafine pigment surface coated with stearic acid, (available from OMYA, Inc.) which also has a top cut particle size of about 4 microns and a mean particle size of about 0.7 microns.

The filler may also include superabsorbent particles such as finely ground sodium polyacrylate or other superabsorbent materials. The superabsorbent materials may provide for absorption of fluids. Additionally, the superabsorbent materials can expand into the void spaces provided by the corrugations 20 to improve fluid wetting, fluid retention, fluid absorption and distribution properties.

The superabsorbent material can be selected from natural, synthetic and modified natural polymers and materials. The superabsorbent material can be inorganic materials, such as silica gels, or organic compounds, such as crosslinked polymers. The term “crosslinked” refers to any means for effectively rendering normally water-soluble materials substantially water insoluble, but swellable. Such means can comprise, for example, physical entanglement, crystalline domains, covalent bonds, ionic complexes and associations, hydrophilic associations, such as hydrogen bonding, and hydrophobic associations or Van der Waals forces. The superabsorbent material can also be modified, such as by surface treating with a cross-linking, substantially non-covalently bonded surface coating with a partially hydrolysable cationic polymer, such as that disclosed in U.S. patent application Ser. No. 10/631,916 entitled “Absorbent Materials And Absorbent Articles Incorporating Such Absorbent Materials” filed Jul. 31, 2003 by Qin et al., which is incorporated herein by reference in a manner that is consistent with the present disclosure.

Superabsorbent materials suitable for use in the present invention are known to those skilled in the art. Processes for preparing synthetic, absorbent gelling polymers are disclosed in U.S. Pat. No. 4,076,663, to Masuda et al. and U.S. Pat. No. 4,286,082, to Tsubakimoto et al., all of which are incorporated herein by reference in a manner that is consistent with the present disclosure. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, chitosan salt, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyvinyl amines, polyquaternary ammonium, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are desirably lightly crosslinked to render the material substantially water insoluble. Crosslinking may, for example, be by irradiation or covalent, ionic, Van der Waals, or hydrogen bonding. Suitable base superabsorbent materials are available from various commercial vendors, such as Stockhausen, Inc., BASF Inc. and others. In one particular aspect, the superabsorbent material can be FAVOR SXM 9394, available from Stockhausen, Inc., a business having offices located in Greensboro, N.C., U.S.A.

As mentioned above, surfactants may also be utilized. Surfactants can increase the hydrophilicity and wettability of the film, can enhance the water vapor permeability of the film, and may improve filler dispersion in the polymer. For example, the surfactant can be blended with the microlayer polymers 16,18 or otherwise incorporated onto the particulate filler material before the filler material is mixed with the polymers. In some aspects, suitable surfactants may have a hydrophile-lipophile balance (HLB) number from about 6 to about 18, such as about 8 to about 16, or about 12 to about 15 to enable microlayer wettability by aqueous fluids. If the HLB number is too low, the wettability may be insufficient, and if the HLB number is too high, the surface active material may have insufficient adhesion to the polymer matrix of the microlayers 16,18, and may be too easily washed away during use. The surfactant modification, or treatment, of the microlayer film 10 or the components of the microlayer film may provide a water contact angle of less than 90 degrees. Desirably, surfactant utilization may provide a water contact angle of less than about 70 degrees. For example, incorporation of DOW CORNING 193 surfactant into the film components may provide a water contact angle of about 40 degrees. A number of suitable commercially available surfactants may be found in McMutcheon's Vol. 2; Functional Materials, 1995.

Suitable surface-active materials for blending with the polymeric components of the microlayer film or treating the particulate filler material include silicone glycol copolymers, ethylene glycol oligomers, acrylic acid, hydrogen-bonded complexes, carboxylated alcohol, ethoxylates, various ethoxylated alcohols, ethoxylated alkyl phenols, ethoxylated fatty esters, stearic acid, behenic acid, and the like, as well as combinations thereof. Examples include surfactants composed of ethoxylated alkyl phenols, such as IGEPAL RC-620, RC-630, CA-620, 630, 720, CO-530, 610, 630, 660, 710, and 730 (available from Rhone-Poulenc, Inc., a business having offices located in Cranbury, N.J., U.S.A.; surfactants composed of silicone glycol copolymers, such as DOW CORNING D190, D193, FF400, and D1315 (available from Dow Corning); surfactants composed of ethoxylated mono and diglycerides, such as MAZOL 80 MGK, MASIL SF 19, and MAZOL 165 C (available from PPG Industries, a business having offices located in Gurnee, Ill., U.S.A.); surfactants composed of ethoxylated alcohols, such as GENAPOL 26-L-98N, GENAPOL 26-L60N, and GENAPOL 26-L-5 (available from Hoechst Celanese Corporation, a business having offices located in Charlotte, N.C., U.S.A.); surfactants composed of carboxylated alcohol ethoxylates, such as MARLOWET 4700 and MARLOWET 4703 (available from Huls America, Inc., a business having offices located in Piscataway, N.J., U.S.A.); and ethoxylated fatty esters, such as PATIONIC 138C, PATIONIC 122A, PATIONIC SSL (available from R.I.T.A. Corporation, a business having offices located in Woodstock, Ill., U.S.A.) The surface activate material is suitably present in the respective microlayer in an amount from about 0.5 to about 20% by weight of the microlayer, such as about 1 to about 15% by weight of the microlayer, or about 2 to about 10% by weight of the microlayer. The surface activate material may be suitably present on the particulate materials in an amount of from about 1 to about 12% by weight of the filler material. The surfactant may be blended with suitable polymers to form a concentrate. The concentrate may then be mixed or blended with polymers forming one or both of the microlayers. In one example, a mixture of molten polypropylene and liquid MASIL SF 19 was blended and allowed to cool into pellets having a surfactant add-on level of 1.5% by weight. The polypropylene/surfactant pellets were then added to the polypropylene resin stream of a coextruder at a ratio of 85 (PP) to 15 (PP/surfactant) to form a low-recovery polymer microlayer 16.

In other aspects, fluid modifiers may also be utilized in the absorbent core 50 of the invention. Suitable fluid modifiers include precipitants, blood coagulants, liquid thickeners or thinners, demulsifiers, buffer agents, inhibitors and the like. Suitable odor control particles include baking soda powder or activated carbon powder. Suitable cooling agents include any endothermic compounds, such as sodium acetate trihydrate (NaC2H3O2.3H2O) and potassium nitrate (KNO3). Suitable heating agents include any exothermic compounds, such as lithium chloride (LiCl) and sodium acetate (NaC2H3O2). Suitable anti-microbial agents (such as bacteriacides, fungicides and the like) include any cationic polymers, such as chitosan, or special surfactants.

In still other aspects, lubricating and release agents may facilitate the formation of micro-voids and the development of a porous structure in the film during stretching of the disrupted portion and may reduce adhesion and friction at filler-resin interface. In addition, odor control agents can help the user feel fresh and confident.

In yet other aspects, nanoparticles may also be utilized in the absorbent core 50. Nanoparticles have particle sizes ranging from 1 to 1000 nanometers. Examples of suitable nanoparticles include titanium dioxide, layered clay minerals, alumina oxide, silicates, and combinations thereof. Other suitable nanoparticles include nano-superabsorbent, nano-complexes for skin care, nanoparticles for odor control, nanoparticles or nanostructures for modifying fluids or as fluid modifiers, nanostructures that create color without dye, nanoparticles that make automotive finishes more scratch resistant, nanoparticles that provide a lotus effect, and the like.

The multi-microlayer film 10 of the present invention may be post-processed to stabilize the film structure in the disrupted portion 32. The post-processing may be accomplished through thermal point or pattern bonding, by embossing, by calendering, by sealing the edges of the film using heat or ultrasonic energy, or by other operations known in the art. Additionally, one or more nonwoven webs may be laminated to the film 10 to improve the strength, tactile properties, appearance, and/or other beneficial properties of the film 10. The nonwoven webs may be spunbond webs, meltblown webs, bonded-carded-webs, airlaid or wetlaid webs, or other nonwoven webs known in the art.

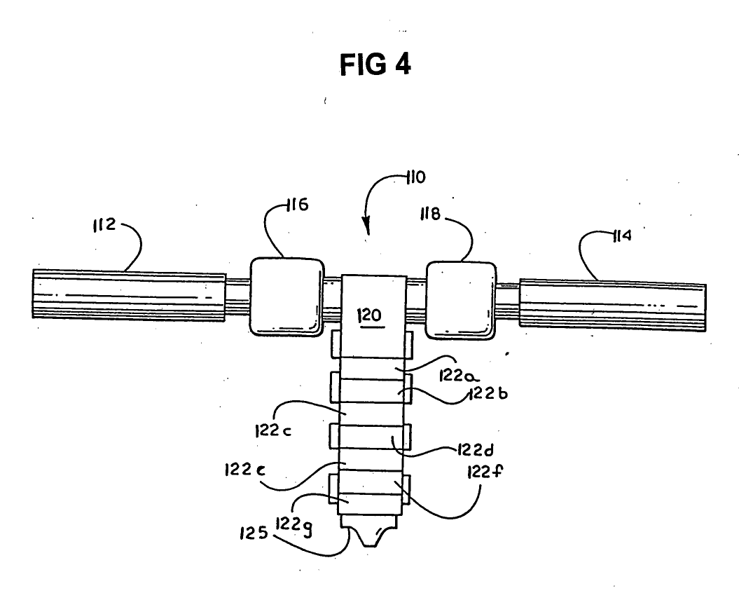

The microlayer film 10 can be made using coextrusion processes known in the art. One suitable method for making the microlayer film of this invention is a microlayer coextrusion process wherein two or more polymers are coextruded to form a laminate with two or more layers, which is then manipulated to multiply the number of layers in the film. FIG. 4 illustrates one such coextrusion device 110 for forming multi-microlayer films. This device includes a pair of opposed single-screw extruders 112,114 connected through respective metering pumps 116,118 to a coextrusion block 120. A plurality of multiplying elements 122a-g extend in series from the coextrusion block 120 perpendicularly to the single-screw extruders 112,114. Each of the multiplying elements 122a-g includes a die element 124 disposed in the melt flow passageway of the coextrusion device 110. The last multiplying element 122g is attached to a discharge nozzle 125 through which the final product extrudes. While single-screw extruders are shown, the present invention may also use twin-screw extruders to form the films of the present invention.

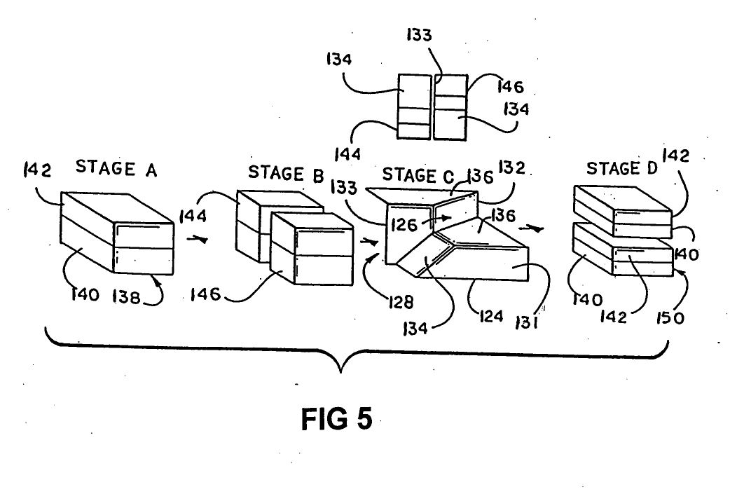

A schematic diagram of the coextrusion process carried out by the coextrusion device 110 is illustrated in FIG. 5. FIG. 5 also illustrates the structure of the die element 124 disposed in each of the multiplying elements 122a-g. Each die element 124 divides the melt flow passage into two passages 126,128 with adjacent blocks 131,132 separated by a dividing wall 133. Each of the blocks 131,132 includes a ramp 134 and an expansion platform 136. The ramps 134 of the respective die element blocks 131 and 132 slope from opposite sides of the melt flow passage toward the center of the melt flow passage. The expansion platforms 136 extend from the ramps 134 on top of one another.

To make a corrugated microlayer film using the coextrusion device 110 illustrated in FIG. 4 and FIG. 5, a high-recovery polymer resin is extruded through the first single screw extruder 112 into the coextrusion block 120. Likewise, a low-recovery polymer, such as polylactic acid or polypropylene, is extruded through the second single screw extruder 114 into the same coextrusion block 120. In the coextrusion block 120, a two-layer melt laminate structure 138, such as that illustrated at stage A in FIG. 5, is formed with the low-recovery polymer forming a layer on top of a layer of the high-recovery polymer.

The melt laminate is then extruded through the series of multiplying elements 122a-g to form a multi-layer microlaminate with the layers alternating between the low-recovery polymer and the high-recovery polymer. As the two-layer melt laminate is extruded through the first multiplying element 122a, the dividing wall 133 of the die element 124 splits the melt laminate 138 into two halves 144,146 each having a layer of low-recovery polymer 140 and a layer of high-recovery polymer 142. This is illustrated at stage B in FIG. 5. As the melt laminate 138 is split, each of the halves 144,146 are forced along the respective ramps 134 and out of the die element 124 along the respective expansion platforms 136.

This reconfiguration of the melt laminate is illustrated at stage C in FIG. 5. When the melt laminate 138 exits from the die element 124, the expansion platform 136 positions the split halves 144,146 on top of one another to form a four-layer melt laminate 150 having a parallel stacking arrangement of a high-recovery polymer layer, a low-recovery polymer layer, a high-recovery polymer layer and a low-recovery polymer layer in laminate form. This process is repeated as the melt laminate proceeds through each of the multiplying elements 122b-g. When the melt laminate is discharged through the discharge nozzle 125, the melt laminate forms a film having from about 4 to about 100 microlayers, depending on the number of multiplying elements.

The foregoing microlayer coextrusion device and process are described in more detail in an article by Mueller et al., entitled Novel Structures By Microlayer Extrusion-Talc-Filled PP, PC/SAN, and HDPE-LLDPE, Polymer Engineering and Science, Vol. 37, No. 2, 1997, which is incorporated herein by reference in a manner that is consistent herewith. A similar process is described in U.S. Pat. No. 3,576,707 and U.S. Pat. No. 3,051,453, each of which is incorporated herein by reference in a manner that is consistent herewith. Other processes known in the art to form multi-microlayer films may also be employed, such as the coextrusion processes described in the article by W. J. Schrenk and T. Ashley, Jr. entitled Coextruded Multilayer Polymer Films and Sheets. Polymer Blends, Vol. 2, Academic Press, New York (1978), which is incorporated herein by reference in a manner that is consistent herewith.

The relative thickness of the microlayers made by the foregoing process can be controlled by varying the feed ratio of the polymers into the extruders, thus controlling the constituent volume fraction. In addition, one or more extruders may be added to the coextrusion device to increase the number of different polymers in the multi-microlayer film. For example, a third extruder may be added to add a tie layer to the film.

It also may be desirable to position the multi-microlayer film 10 within an absorbent article such that the disrupted portion 32 is located within an insult target zone of the article. Because of the increased bulk and void spaces 22 formed by corrugations 20, the disrupted portion 32 has the capability of performing the multiple functions of an acquisition layer, a distribution layer and a retention layer.

The absorbent core 50 of the present invention can optionally comprise an additional retention layer. The multi-microlayer film 10 of the present invention may be placed in planar contact with and adjacent to, and/or may be bonded to, the retention layer using means known in the art. The retention layer may comprise absorbent material, such as superabsorbent material and/or fluff. Additionally, the superabsorbent material can be operatively contained within a matrix of fibers, such as polymeric fibers, such as elastomeric polymer fibers. In some aspects, the amount of superabsorbent material in the retention layer can be at least about 10% by weight of the layer, such as at least about 30%, or at least about 60% by weight or at least about 80%, or between about 10% and about 80% by weight of the retention layer to provide improved benefits. Optionally, the amount of superabsorbent material can be at least about 95% by weight of the retention layer. In other aspects, the retention layer can comprise about 35% or less by weight fluff, such as about 25% or less, or 15% or less by weight fluff.

It should be understood that the retention layer is not restricted to use with superabsorbent materials and/or fluff. In some aspects, the retention layer may additionally or alternatively include materials such as surfactants, ion exchange resin particles, moisturizers, emollients, perfumes, natural fibers, synthetic fibers, fluid modifiers, odor control additives, and combinations thereof. Alternatively, the retention layer can be or can include a foam.

When utilized with a retention layer, it may be particularly desirable to aperture both portions 32,34 of the multi-microlayer film 10. Apertures which penetrate through the disrupted portion 32 of the multi-microlayer film 10 can channel a quantity of the fluids directly into the retention layer at a more central location, while apertures in the non-disrupted portion 32 can provide avenues for fluids to exit in more remote locations of the optional retention layer.

The absorbent core 50 of the present invention may also be attached or laminated to one or more nonwoven webs. The nonwoven webs may be spunbond webs, meltblown webs, bonded-carded-webs, airlaid or wetlaid webs, or other nonwoven webs known in the art.

Attachment or lamination may be accomplished using methods known in the art, such as thermal or adhesive bonding. Thermal bonding may be accomplished by, for example, point bonding. Adhesive bonding may be accomplished by, for example, melt spraying, printing or meltblowing an adhesive material to at least one of the components. Various types of adhesives are available including those produced from amorphous polyalphaolefins and ethylene vinyl acetate-based hot melts.

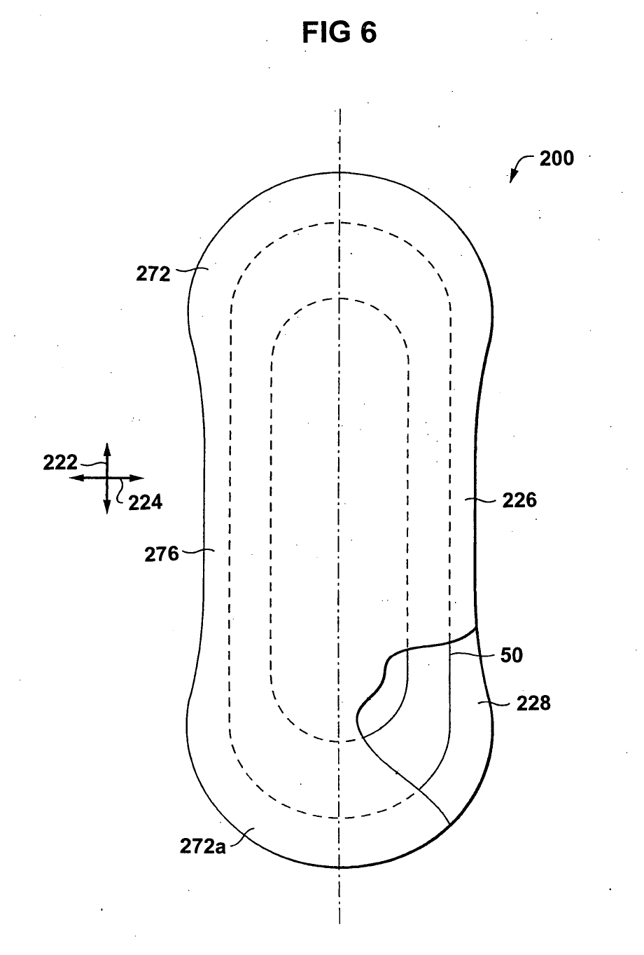

FIG. 6 illustrates an example of a suitable article, such as the representatively shown feminine care article 200, which is configured to incorporate the present invention. The feminine care article can have a lengthwise longitudinal direction 222, a transverse, laterally extending, cross-direction 224, first and second longitudinally opposed end portions 272 and 272a, and an intermediate portion 276 located between the end portions. As representatively shown, the longitudinal dimension of the article is relatively larger than the lateral dimension of the article. The article 200 can include a top sheet or cover 226, a baffle 228, and the absorbent core 50 of the present invention positioned between the cover and baffle. The absorbent core 50 can have configurations which are selectively constructed and arranged to provide desired performance and aesthetics.

By incorporating its various features, aspects and configurations, alone or in desired combinations, the article can provide an improved absorbent system that can take better advantage of the functional properties of the absorbent core 50 of the present invention. For example, the article can provide comparable or improved absorbent properties when compared to an article comprising a conventional absorbent core. Similarly, the article can provide comparable or improved absorbent properties while reducing the thickness of the article, as well as improved production costs, when compared to an article comprising a conventional absorbent core.

The absorbent core 50 can also distribute viscous fluids more efficiently to desired locations in an absorbent article, as well as provide a drier body-facing surface, and in particular configurations, can provide visual cues of absorbency. Other examples can provide improved appearance and aesthetics. As a result, an article incorporating the invention can provide greater comfort and fit, and can improve protection and increase confidence.

The topsheet 226 of an absorbent article 200 which includes the absorbent core 50 of the present invention may include a layer constructed of any operative material, and may be a composite material. For example, the topsheet layer can include a woven fabric, a nonwoven fabric, a polymer film, a film-fabric laminate or the like, as well as combinations thereof. Examples of a nonwoven fabric include spunbond fabric, meltblown fabric, coform fabric, carded-web, bonded-carded-web, bicomponent spunbond fabric or the like, as well as combinations thereof. For example, the topsheet layer can include a woven fabric, a nonwoven fabric, a polymeric film that has been configured to be operatively liquid-permeable, or the like, as well as combinations thereof. Other examples of suitable materials for constructing the topsheet layer can include rayon, bonded-carded-webs of polyester, polypropylene, polyethylene, nylon, or other heat-bondable fibers, polyolefins, such as copolymers of polypropylene and polyethylene, linear low-density polyethylene, biodegradable aliphatic polyesters such as poly(hydroxyl alkanoates) and polyactic acid, finely perforated film webs, net materials, and the like, as well as combinations thereof.

A more particular example of a suitable topsheet layer material can include a bonded-carded-web composed of polypropylene and polyethylene, such as has been used as a topsheet stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. In a desired arrangement, the topsheet layer 226 can be configured to be operatively liquid-permeable with regard to the liquids that the article is intended to absorb or otherwise handle. The operative liquid-permeability may, for example, be provided by a plurality of pores, apertures or other openings, as well as combinations thereof, that are present or formed in the topsheet layer. The apertures or other openings can help increase the rate at which bodily fluids can move through the thickness of the topsheet layer and penetrate into the other components of the article (e.g., the absorbent core 50). The selected arrangement of liquid-permeability is suitably present at least on an operative portion of the topsheet layer that is appointed for placement on the body-side of the article. The topsheet layer 226 can provide comfort and conformability, and can function to direct viscous fluids, such as menses, away from the body and toward the absorbent core 50. In one example, the topsheet layer 226 can be configured to retain little or no liquid in its structure, and can be configured to provide a relatively comfortable and non-irritating surface next to the body-tissues of the wearer. The topsheet layer 226 can be constructed of any material which is also easily penetrated by viscous fluids that contact the surface of the topsheet layer.

The topsheet 226 can have at least a portion of its bodyside surface treated with a surfactant to render the topsheet more hydrophilic. The surfactant can permit arriving viscous liquids to more readily penetrate the topsheet layer. The surfactant may also diminish the likelihood that the arriving viscous fluids, such as menses, will flow off the topsheet layer rather than penetrate through the topsheet layer into other components of the article. In one example, the surfactant can be substantially evenly distributed across at least a portion of the upper, bodyside surface of the topsheet 226 that overlays the body-facing surface of the absorbent core 50.

The topsheet 226 may be maintained in secured relation with the absorbent core 50 of the present invention by bonding all or a portion of the adjacent surfaces to one another. A variety of bonding techniques known to one of skill in the art may be utilized to achieve any such secured relation. Examples of such techniques include, but are not limited to, the application of adhesives in a variety of patterns between the two adjoining surfaces, entangling at least portions of the adjacent surface of the absorbent core 50 with portions of the adjacent surface of the topsheet 226, co-aperturing or fusing at least portions of the adjacent surface of the topsheet 226 to portions of the adjacent surface of the absorbent core 50.

The topsheet 226 typically extends over the body-facing surface of the absorbent core 50, but can alternatively extend around the article to partially, or entirely, surround or enclose the core. Alternatively, the topsheet 226 and the backsheet 228 can have peripheral margins which extend outwardly beyond the terminal, peripheral edges of the absorbent core 50, and the extending margins can be joined together to partially, or entirely, surround or enclose the core. In some aspects, the multi-microlayer film of the present invention can function as the topsheet, in addition to functioning as an absorbent core. Thus the multi-microlayer film can also provide an innermost bodyside surface.

The backsheet 228 may include a layer constructed of any operative material, and may or may not have a selected level of liquid-permeability or liquid-impermeability, as desired. In one example, the backsheet or backsheet 228 may be configured to provide an operatively liquid-impermeable backsheet structure. The backsheet 228 may, for example, include a polymeric film, a woven fabric, a nonwoven fabric or the like, as well as combinations or composites thereof. For example, the backsheet 228 may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, have a printed message to the consumer, and/or may be at least partially colored. Suitably, the backsheet 228 can operatively permit a sufficient passage of air and moisture vapor out of the article, particularly out of the absorbent core 50, while blocking the passage of bodily liquids.

An example of a suitable backsheet material can include a breathable, microporous film, such as a HANJIN Breathable Backsheet available from Hanjin Printing, Hanjin P&C Company Limited, a business having offices located in Sahvon-li.Jungan-mvu.Kongiu-City, Chung cheong nam-do, Republic of South Korea. This backsheet material is a breathable film, which is white in color, dimple embossed, and contains: 47.78% calcium carbonate, 2.22% TiO2, and 50% polyethylene.

In one example, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed-cell polyolefin foam. For example, a closed-cell polyethylene foam may be employed. Still another example of a backsheet material would be a material that is similar to a polyethylene film which is used on commercially sold KOTEX brand pantiliners, and is obtainable from Pliant Corporation, a business having offices located in Schaumburg, Ill., U.S.A. In some aspects, the multi-microlayer film of the present invention can function as the backsheet, in addition to functioning as the absorbent core. Thus, the multi-microlayer film can provide an outermost garment-facing surface.

The absorbent core 50 comprising the multi-microlayer film 10 can be sized and placed to more effectively operate in a insult target zone of the absorbent article 200 where liquids are more likely to be introduced into the article. The structure of the absorbent core 50 can be operatively configured to provide a desired level of fluid acquisition, distribution and retention. As described above, the absorbent core 50 may include one or more components that can modify the composition or rheological properties of such viscous fluids. The absorbent core 50 can also include absorbent material, such as superabsorbent material and/or fluff.

The present invention may be better understood with reference to the following examples.

### EXAMPLES

### Example 1

Polypropylene (“PP”) 3155 resin (available from ExxonMobil Chemical Company, a business having offices located in Houston, Tex., U.S.A.) was compounded with 20% by weight molecular sieves and 5% by weight calcium carbonate filler using a ZSK30 twin screw extruder (available from Werner & Pfleiderer, a business having offices located in Tamm, Germany) to form filled PP. The mean particle size of the calcium carbonate was 1 micron. The filled polypropylene was dry blended with a 1.5% by weight surfactant/PP concentrate in a ratio of 85 to 15 for the filled PP to the surfactant/PP concentrate. The surfactant utilized was MANSIL SF-19, available from BASF Corporation. The blend of filled polypropylene and surfactant/PP concentrate was then fed into one extruder of the coextrusion line. The extruder and volumetric pump temperatures for the mixture were set at 185° C.

Pellets of thermoplastic polyurethane elastomer ESTANE 58245 that had been dried over night at 50° C. were then fed into the other extruder of the coextrusion line. The extruder and volumetric pump temperatures for the polyurethane elastomer were set at 210° C.

The elastomer and the PP mixture were processed through the extruders, and a 17-layer laminated multi-microlayer film having a weight ratio of 70 to 30 (elastomer to PP) was produced using four multiplier-die elements set at 175° C. The films were produced with a total thickness of about 0.25 mm and had a layer of the PP mixture on the outer surface of both sides of the multi-microlayer film.

The film was allowed to cool to ambient temperature and was cut into several 5-cm by 45-cm strips (45-cm in the machine direction). Approximately 10 centimeters in a center portion of each strip was then stretched by hand to about 200% strain in the machine direction (MD) at a temperature of about 25° C. The 10-cm portion partially delaminated during the stretching and after releasing the stretching force, the polyurethane microlayers substantially recovered their pre-stretch length, while the PP mixture microlayers recovered very little of their pre-stretch length, thus forming corrugations in the stretched portion of the film. The thickness of the stretched portion as measured at 0.05 psi using a MITUTOYO DIGIMATIC INDICATOR (available from Mitutoyo America Corporation, a business having offices located in Aurora, Ill., U.S.A.) was approximately 6 mm, while the remainder of the multi-microlayer film remained laminated and unstretched, retaining its original thickness of 0.25 mm. The 10-cm partially delaminated portion was about 14 cm in length after stretching. This section was then apertured by hand using a 2-mm punch to create 10 holes that penetrated all of the way through the layers.

Each sample was placed in a cradle test stand, such as described in U.S. Pat. No. 5,843,063 to Anderson et al., except that the width of the stand was reduced to 3.5 cm and the stand did not have a slotted opening on the bottom. The stretched portion of the sample was then insulted with 50 grams of 0.9% by weight saline over a time period of about 6 minutes. Then a second insult with 50 grams of the saline was conducted in a similar manner. After 60 minutes, it was observed that the fluid had wicked to each end of the film, about 24 cm from the lowest point of the film. It was also observed that the fluid movement resulted in a partial debonding of the unstretched portions of the film, such that the final thickness increased from 0.25 mm to about 0.9 mm at the highest point.

This example demonstrates that through film capillary actions, the disrupted portion of the multi-microlayer film carried out the functions of fluid acquisition, retention and distribution, while the non-disrupted portion carried out additional retention and distribution.

### Example 2

Polypropylene (“PP”) 3155 resin (available from ExxonMobil Chemical Company) was compounded with 20% by weight molecular sieves and 5% by weight calcium carbonate filler using a ZSK30 twin screw extruder (available from Werner & Pfleiderer) to form filled PP. The mean particle size of the calcium carbonate was 1 micron. The filled polypropylene was dry blended with a 1.5% by weight surfactant/PP concentrate in a ratio of 85 to 15 for the filled PP to the surfactant/PP concentrate. The surfactant utilized was MANSIL SF-19, available from BASF Corporation. The blend of filled polypropylene and surfactant/PP concentrate was then fed into one extruder of the coextrusion line. The extruder and volumetric pump temperatures for the mixture were set at 185° C.

Pellets of thermoplastic polyurethane elastomer ESTANE 58245 that had been dried over night at 50° C. were then fed into the other extruder of the coextrusion line. The extruder and volumetric pump temperatures for the polyurethane elastomer were set at 210° C.

The elastomer and the PP mixture were processed through the extruders, and a 17-layer laminated multi-microlayer film having a weight ratio of 70 to 30 (elastomer to PP) was produced using four multiplier-die elements set at 175° C. The films were produced with a total thickness of about 0.12 mm and had a layer of the PP mixture on the outer surface of both sides of the multi-microlayer film.

The films were allowed to cool to ambient temperature and were cut into several 4.5-cm by 31-cm strips (31-cm in the machine direction). Approximately 2 centimeters in a center portion of each strip was then stretched by hand to about 150% strain in the machine direction (MD) at a temperature of about 35° C. The 10-cm portion partially delaminated during the stretching and after releasing the stretching force, the polyurethane microlayers substantially recovered their pre-stretch length, while the PP mixture microlayers recovered very little of their pre-stretch length, thus forming corrugations in the stretched portion of the film. The thickness of the stretched portion as measured at 0.05 psi using a MITUTOYO DIGIMATIC INDICATOR was approximately 5 mm, while the remainder of the multi-microlayer film remained laminated and unstretched, retaining its original thickness of 0.12 mm. The 2-cm partially delaminated portion was about 2.5 cm in length after stretching. This section was then apertured by hand using a 2-mm punch to create 2 holes that penetrated all of the way through the layers.

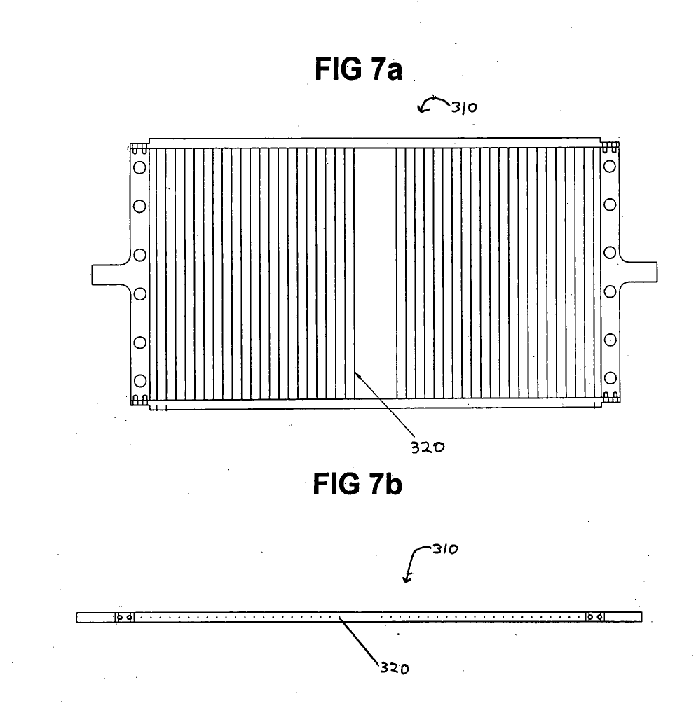

With reference to FIG. 7, each sample was placed on a Horizontal Wick Test Bed 310 with the 2.5-cm delaminated portion centered above a fluid addition device. The test bed 310 measured approximately 33 cm in length by 16 cm in width, and had a series of poly strands 320 spaced approximately 0.64 cm apart and stretched taught across the width of the test bed 310. The test bed 310 was placed into an X-ray unit (not shown) and fluid flow was started. X-rays were taken at elapsed times of 1,2,5,10, and 20 minutes. Then a second 20-minute fluid insult and wicking test was performed on the same sample, with measurements taken at the same elapsed time.

This example demonstrates that through film capillary actions, the disrupted portion of the multi-microlayer film carried out the functions of fluid acquisition, retention and distribution, while the non-disrupted portion carried out additional retention and distribution by moving fluid to the end of the film in 20 minutes to a level of at least 8 g/g and within 40 minutes to a level of 18 g/g.

What is claimed is:

1. An absorbent core comprising:
a multi-microlayer film having a plurality of coextruded laminated microlayers including a microlayer of a high-recovery polymer and a microlayer of a low-recovery polymer;
wherein the multi-microlayer film has a disrupted portion where the microlayer of a low-recovery polymer is at least partially delaminated from the microlayer of a high-recovery polymer; and
wherein the disrupted portion comprises apertures.

2. The absorbent core of claim 1 wherein the microlayer of a low-recovery polymer is gathered in the disrupted portion.

3. The absorbent core of claim 1 wherein the microlayer of a low-recovery polymer is plastically deformed in the disrupted portion.

4. The absorbent core of claim 1 wherein the microlayer of a high-recovery polymer comprises a thermoplastic elastomeric polymer.

5. The absorbent core of claim 4 wherein the thermoplastic elastomer is selected from metallocene polyolefins, single-site catalyzed olefinic elastomers, thermoplastic polyester, thermoplastic polyether polyurethanes, EVA copolymers, polyether block polyamide copolymers, ether-ester segmented-block elastomers, styrenic block copolymers, or combinations thereof.

6. The absorbent core of claim 1 wherein the microlayer of a low-recovery polymer comprises a thermoplastic stretchable polymer.

7. The absorbent core of claim 6 wherein the thermoplastic stretchable polymer is selected from polycaprolactone, polymers and copolymers of polylactic acid, polymers and copolymers of polybutylene succinate, poly(butylene succinate-adipate), polyesters, polypropylene, copolymers of polypropylene, polyethylene, copolymers of polyethylene, or combinations thereof.

8. The absorbent core of claim 1 wherein the microlayer of a low-recovery polymer further comprises an additional material selected from a filler material, a surfactant, a blowing agent, a superabsorbent material, a fluid modifier, or combinations thereof.

9. The absorbent core of claim 8 wherein the filler material is a particulate material selected from molecular sieves, zeolites, metal oxides, metal hydroxides, metal carbonates, metal sulfates, clays, silica, alumina, powdered metals, glass microspheres, calcium carbonate, barium sulfate, sodium carbonate, magnesium carbonate, magnesium sulfate, barium carbonate, kaolin, carbon, calcium oxide, magnesium oxide, aluminum hydroxide, titanium dioxide, talc, mica, wollastonite, latex particles, particles of thermoplastic elastomers, pulp powders, wood powders, thermoplastic starch, carboxymethyl cellulose, hydroxypropyl cellulose, chitin, chitosan powder, organosilicone powders, polyacrylic acid, or combinations thereof.

10. The absorbent core of claim 1 wherein the microlayer of a high-recovery polymer further comprises an additional material selected from a filler material, a surfactant, a blowing agent, a superabsorbent material, a fluid modifier, or combinations thereof.

11. The absorbent core of claim 10 wherein the filler material is a particulate material selected from molecular sieves, zeolites, metal oxides, metal hydroxides, metal carbonates, metal sulfates, clays, silica, alumina, powdered metals, glass microspheres, calcium carbonate, barium sulfate, sodium carbonate, magnesium carbonate, magnesium sulfate, barium carbonate, kaolin, carbon, calcium oxide, magnesium oxide, aluminum hydroxide, titanium dioxide, talc, mica, wollastonite, latex particles, particles of thermoplastic elastomers, pulp powders, wood powders, thermoplastic starch, carboxymethyl cellulose, hydroxypropyl cellulose, chitin, chitosan powder, organosilicone powders, polyacrylic acid, or combinations thereof.

12. The absorbent core of claim 1 wherein each microlayer has a thickness of from about 0.1 micron to about 100 microns.

13. The absorbent core of claim 1 wherein a non-disrupted portion of the multi-microlayer film has a thickness measured at 0.05 psi of about 5 millimeters or less.

14. The absorbent core of claim 1 wherein the multi-microlayer film comprises from about 4 to about 100 microlayers.

15. The absorbent core of claim 1 wherein the disrupted portion has a thickness measured at 0.05 psi of at least 5 times the thickness of a non-disrupted portion.

16. The absorbent core of claim 1 wherein the microlayers of a low-recovery polymer in the disrupted portion have corrugations which provide channel spaces oriented in the cross-machine direction; and
wherein the microlayers of a high-recovery polymer in the disrupted portion have corrugations which provide channel spaces oriented in the machine direction.

17. The absorbent core of claim 1 wherein the disrupted portion is located in an insult target zone of an absorbent article.

18. The absorbent core of claim 1 wherein a non-disrupted portion of the multi-microlayer film comprises apertures.

19. The absorbent core of claim 1 further comprising a retention layer positioned adjacent to and in planar contact with the multi-microlayer film.

20. The absorbent core of claim 1 further comprising a liquid-permeable topsheet and a backsheet, wherein the multi-microlayer film is positioned between the liquid-permeable topsheet and the backsheet.

21. The absorbent core of claim 1 further comprising a liquid-permeable topsheet.

22. The absorbent core of claim 21 wherein the multi-microlayer film provides an outermost garment-facing surface.

23. The absorbent core of claim 1 further comprising a backsheet.

24. The absorbent core of claim 23 wherein the multi-microlayer film provides an innermost bodyside surface.

25. The absorbent core of claim 19 further comprising a liquid-permeable topsheet and a backsheet, wherein the multi-microlayer film is positioned between the liquid-permeable topsheet and the backsheet.

26. The absorbent core of claim 1 wherein said absorbent core is a component of an article selected from personal care absorbent articles, health/medical absorbent articles, and household/industrial absorbent articles.

27. The absorbent core of claim 1 wherein the disrupted portion is apertured prior to stretching.

28. The absorbent core of claim 1 wherein the disrupted portion is apertured with at least 5 apertures per square centimeter.
